(19) United States
(12) Patent Application Publication
(10) Pub. No.: US 2016/0317430 A1
(43) Pub. Date: Nov. 3, 2016

(54) COSMETIC USE OF AN EXTRACT OF GYMNEMA SYLVESTRE

(71) Applicant: LABORATOIRES CLARINS, Neully Sur Seine (FR)

(72) Inventor: Olivier Courtin, Neuilly Sur Seine (FR)

(73) Assignee: Laboratoires Clarins, Neuilly Sur Seine (FR)

(21) Appl. No.: 15/107,630

(22) PCT Filed: Dec. 18, 2014
(86) PCT No.: PCT/IB2014/067067

§ 371 Date: Jun. 23, 2016

(30) Foreign Application Priority Data
Dec. 24, 2013 (FR) 13 63527

(51) Int. Cl. A61K 8/97; A61K 8/34; A61Q 19/08; A61Q 19/00; A61Q 19/04; A61K 36/24; A61K 47/10

(52) U.S. Cl. CPC A61K 8/97; A61K 36/24; A61K 8/345; A61K 47/10; A61Q 19/00; A61Q 19/04; A61Q 19/08; A61K 2236/333; A61K 2236/53; A61K 2800/78; A61K 2800/592

(57) ABSTRACT

The invention relates to the cosmetic use of an extract of Gymnema sylvestre and to cosmetic compositions comprising said extract for increasing the synthesis of ATP, for stimulating the activity of cytochrome c oxidase, for inducing the synthesis of ribose 5-phosphate, and thus for regulating the metabolic and energy processes relating to the skin.

The present invention relates to the field of cosmetic skin treatment.

The skin is a special organ in the human body. It is thin and very extensive as it covers the entire surface of the body and, in adults, has a total surface area of approximately 1.6 m². The function thereof is that of protecting deep tissue from the outside environment, in terms of physical ingress of foreign bodies, and immunity, temperature regulation, fluid loss, etc. Finally, the mechanical stress to which it is continuously subjected means that it requires a strong and cohesive structure associated with great flexibility. Due to the environmental attacks sustained by the skin, it is important to maintain the renewal and repair thereof by stimulating the metabolism of the constituent cells thereof.

Within the scope of its studies on the regulation of metabolic and energy processes in skin cells, the Applicant has revealed the beneficial effect of a Gymnema sylvestre extract on the metabolism of skin cells; it has more particularly demonstrated that this extract can stimulate the mitochondria and increase the cellular energy level and thus make it possible to prevent or combat the signs of skin aging, and also damage caused by external attacks, such as the cold, wind, UV radiation, radiation, or exposures to toxins or pollutants.

These studies have particularly demonstrated the effect of a Gymnema sylvestre extract on:
ATP synthesis in human keratinocytes in culture:

Energy in the cell is produced from basic molecules (carbohydrates, proteins, fats) essentially obtained from the diet. The oxidation of these compounds in the mitochondria gives rise to the release of energy in the form of a high-energy compound, adenosine triphosphate (ATP).

When the cell needs energy, the terminal phosphate group of ATP is hydrolyzed to release the energy from the bond and adenosine diphosphate (ADP). According to the requirements of the cells, ATP is constantly hydrolyzed, and then regenerated. However, with age and the various oxidative stress sustained by skin cells, mitochondrial integrity and function are impaired: ATP neosynthesis is reduced.

It would thus appear to be important to identify agents capable of increasing the intracellular ATP content and boost the metabolism of skin cells by stimulating ATP neosynthesis.

The results presented in the experimental part hereinafter demonstrate that a Gymnema sylvestre extract induces a significant increase in ATP synthesis.
the activity of cytochrome C oxidase, complex IV of the respiratory chain, in human keratinocytes in culture subjected to oxidative stress or not:

Cytochrome C oxidase is found in the various skin and hair cell types; it is known to be involved in the energy metabolism in the mitochondrial respiratory chain or redox chain, which controls the energy level, metabolism and homeostasis of cells. This enzyme is a photoacceptor activated by red light: when photons are absorbed by the redox chain, they transfer the energy thereof to the respiratory system inducing, via the change of electrochemical potential of the photoactivated cell and the transduction of intracellular signals mediated by Ca²⁺ and cAMP, a cascade of events in the cell (DNA and RNA synthesis, proliferative cell activity) and energy production in the form of ATP.

This enzyme is capable of catalyzing the transfer of electrons to molecular oxygen for the conversion thereof into water molecules without forming intermediate free radicals harmful for the cell, according to the following diagram:

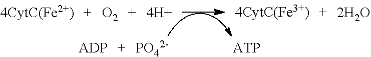

Diagram of Reaction Inducing the Production of ATP and Water Molecules from Oxygen by CytoC Oxidase

Cytochrome C, one of the substrates thereof, is an essential component of the respiratory chain. Cytochrome C plays an essential role in mitochondrial function and in cell survival. The primary role of cytochrome C is to ensure the transfer of electrons, by means of the change of valence of the iron atom. Cytochrome C which is soluble, thus carries the electrons from complex III to complex IV (cytochrome oxidase). The electrons, which are the substrate of cytochrome oxidase, are then transferred by the enzyme to the oxygen.

Numerous data make it possible at the present time to assert that free radicals, by inducing lipid peroxidation reactions, are responsible for a significant proportion of the decrease in membrane fluidity and the fall in activity of some transporters such as cytochrome oxidase, in the mitochondria of aged tissue; therefore, the identification of compounds capable of increasing cytochrome C oxidase activity would prove to be useful.

It was demonstrated that Gymnema sylvestre extract stimulates the cytochrome C oxidase activity of human keratinocytes; in addition, the effect of Gymnema sylvestre extract was tested on human keratinocytes previously treated with hydrogen peroxide (H₂O₂), which has the effect of inducing a significant decrease in cytochrome C oxidase activity; it was then demonstrated that Gymnema sylvestre extract restores cytochrome C oxidase activity significantly.

In conclusion, Gymnema sylvestre extract has a significant effect on the regulation of metabolic and energy processes in human keratinocytes in culture.
the pentose pathway in human fibroblasts in culture by the ribose-5-phosphate assay:

The Applicant's tests also demonstrated that Gymnema sylvestre extract has an effect on the regulation of metabolic and energy processes in human keratinocytes and fibroblasts in culture by inducing a significant increase in ribose-5-phosphate synthesis.

In parallel with the glycolysis which is involved in ATP production, the pentose phosphate pathway is a metabolic pathway which produces NAPDH, an electron donor particularly promoting cell respiration (Krebs cycle, respiratory chain), and ribose-5-phosphate (DNA, RNA biosynthesis).

Due to the properties thereof, Gymnema sylvestre extract thus appears to be a useful agent for increasing the energy metabolism of skin cells.

Gymnema sylvestre is a native plant of the tropical forests of central and southern India and Sri Lanka; this plant is traditionally used in medicinal preparations for various therapeutic uses, in particular for treating diabetes.

It is known to use extracts of Gymnema sylvestre in compositions intended to be applied to the skin, particularly due to the coloring nature of this compound or for the hair growth inhibition properties thereof (WO 2005/067953).

However, to the Applicant's knowledge, it has never been hitherto suggested that a Gymnema sylvestre extract could have the properties cited above and prove to be useful as an activation agent of the metabolic pathway supplying energy to skin cells.

The present invention thus relates to the cosmetic use of a Gymnema sylvestre extract or to a cosmetic composition comprising such an extract for activating the energy-producing metabolic pathways of skin cells; for increasing ATP synthesis; for stimulating cytochrome C oxidase activity; for inducing ribose-5-phosphate synthesis; and thus for regulating the metabolic and energy processes in the skin.

Gymnema sylvestre extract is preferably obtained from the aerial part of the plant, more preferentially from the leaves, and may be prepared using any extraction method known to those skilled in the art.

According to one particular embodiment, the extraction is carried out using fragments of aerial parts of Gymnema sylvestre by the following treatment:
drying followed by grinding;
solid/liquid extraction in hydroalcoholic phase, for example in a mixture of water and C₁ to C₄ lower alcohol such as ethanol;
coarse filtration;
discoloration on activated carbon;
plate filtration with a 1 μm cut-off threshold;
concentration.

To facilitate the subsequent use thereof, the extract may then be solubilized on a cosmetically acceptable substrate, such as glycerin.

A clear amber liquid extract is thus obtained.

Advantageously, Gymnema sylvestre extract is a hydroalcoholic extract and/or is stabilized with glycerin.

For the cosmetic use thereof, Gymnema sylvestre extract is formulated in a cosmetic composition comprising between 0.01 and 10%, preferably between 0.01 and 5%, of Gymnema sylvestre extract by weight of dry matter with respect to the total weight of the composition.

This composition may be presented in any of the dosage forms normally used in the cosmetic and dermatological fields, and it may notably be in the form of an optionally gelled aqueous solution, an optionally bi-phase lotion type dispersion, an emulsion obtained by dispersing an oil phase in an aqueous phase (O/W) or conversely (W/O), or a triple emulsion (W/O/W or O/W/O) or a vesicle dispersion of the ionic and/or non-ionic type. These compositions are prepared using the usual methods.

This composition may be more or less fluid and have the appearance of a white or colored cream, ointment, milk, lotion, serum, paste or foam. It may optionally be applied on the skin in aerosol form. It may also be presented in solid form, particularly in stick form. It may be used as a skin care product and/or makeup product.

The composition according to the invention may also contain the usual adjuvants in the cosmetic field, such as hydrophilic or lipophilic gelling agents, hydrophilic or lipophilic agents, preservatives, antioxidants, solvents, fragrances, bulking agents, filters, pigments, odor absorbers and dyes. The quantities of these different adjuvants are those conventionally used in the field in question, and for example from 0.01 to 20% of the total weight of the composition. These adjuvants, according to the nature thereof, may be introduced into the oily phase, into the aqueous phase, into lipid vesicles and/or into nanoparticles. In any case, these adjuvants, along with the proportions thereof, will be chosen so as not to impede the sought properties of Gymnema sylvestre extract according to the invention.

According to the intended use of the composition according to the invention, Gymnema sylvestre extract may be associated with further anti-aging agents, in particular anti-wrinkle, depigmenting, firming, draining, tightening, anti-radical and/or immunoprotective agents and/or agents stimulating the cell metabolism (particularly protein synthesis) and/or cell proliferation.

The Applicant demonstrated that Gymnema sylvestre extract, and as such the cosmetic composition according to the invention containing same, may be used for activating the metabolic pathways of skin cells, in particular those supplying cell energy. The term skin cells denotes keratinocytes, fibroblasts and melanocytes; preferably, it consists of keratinocytes and fibroblasts.

Consequently, the use of a Gymnema sylvestre extract is an agent for toning skin cells and/or stimulating the skin cell metabolism; this extract and/or the cosmetic composition according to the invention may thus be used as an agent for toning skin cells and/or stimulating the skin cell metabolism.

Gymnema sylvestre extract and/or the cosmetic composition according to the invention may further be used to enable optimal function of skin cells, ensure the homeostasis, renewal and repair thereof.

As such, Gymnema sylvestre extract and/or the composition according to the invention are advantageously used for energizing, toning, revitalizing or detoxifying the skin, in particular stressed and tired skin, by enhancing the radiance of the complexion thereof and the appearance of the surface thereof; they are also used for preventing and/or repairing the signs of fatigue on the face such as a dull and/or gray complexion; they are also used for protecting skin cells and the skin from aggressive environmental factors, such as weather-related factors, like the cold or the wind, or pollution.

Gymnema sylvestre extract and/or the composition according to the invention are also useful for enhancing the color and/or radiance of the complexion of the skin; as such, they prevent the appearance of dull and/or lackluster skin.

Gymnema sylvestre extract and/or the composition according to the invention are further useful for toning the contour of the eyes, energizing tired eyes, reducing bags and shadows under the eyes and generally restoring a relaxed and rested expression.

Gymnema sylvestre extract and/or the composition according to the invention may be used in energizing or anti-tiredness treatments.

The applications above preferentially relate to facial skin, i.e. the skin on the face, neck, décolleté and/or eye contour but are also suitable for cosmetic treatments specific for skin on the body.

The present invention also relates to a cosmetic treatment method for activating the metabolic pathways supplying energy to skin cells; energizing, toning, revitalizing or detoxifying the skin, and toning the contour of the eyes, energizing tired eyes, reducing bags and shadows under the eyes and generally restoring a relaxed and rested expression; comprising the application of the skin of a Gymnema sylvestre extract or a cosmetic composition comprising a Gymnema sylvestre extract as described above.

Application on the skin may thus be carried out according to the dosage form used. It may consist, for example, of merely an application on the skin or an application accompanied by a massage of the skin with the composition according to the present invention.

Application is preferably carried out with a sufficient quantity of the composition so that the entire surface of the skin to be treated is treated. It may consist for example of a conventional application, such as a cream on the skin. It may also consist for example also of an application to act as a treating mask.

The examples hereinafter relate, on one hand, to the evaluation of the biological effects of a Gymnema sylvestre extract and, on the other, of compositions according to the present invention.

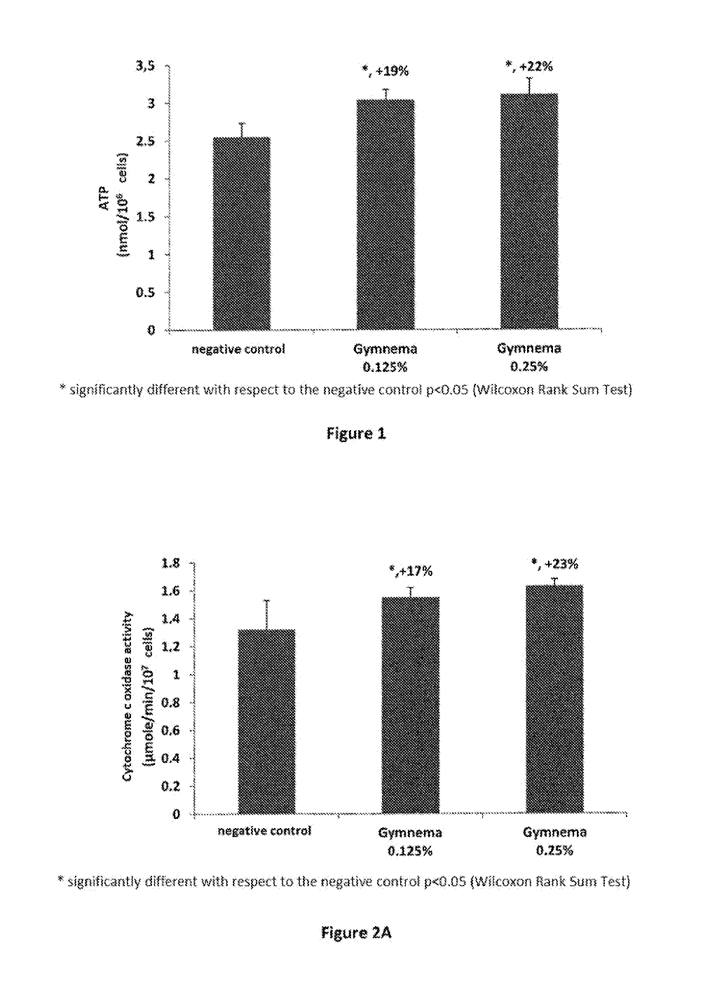

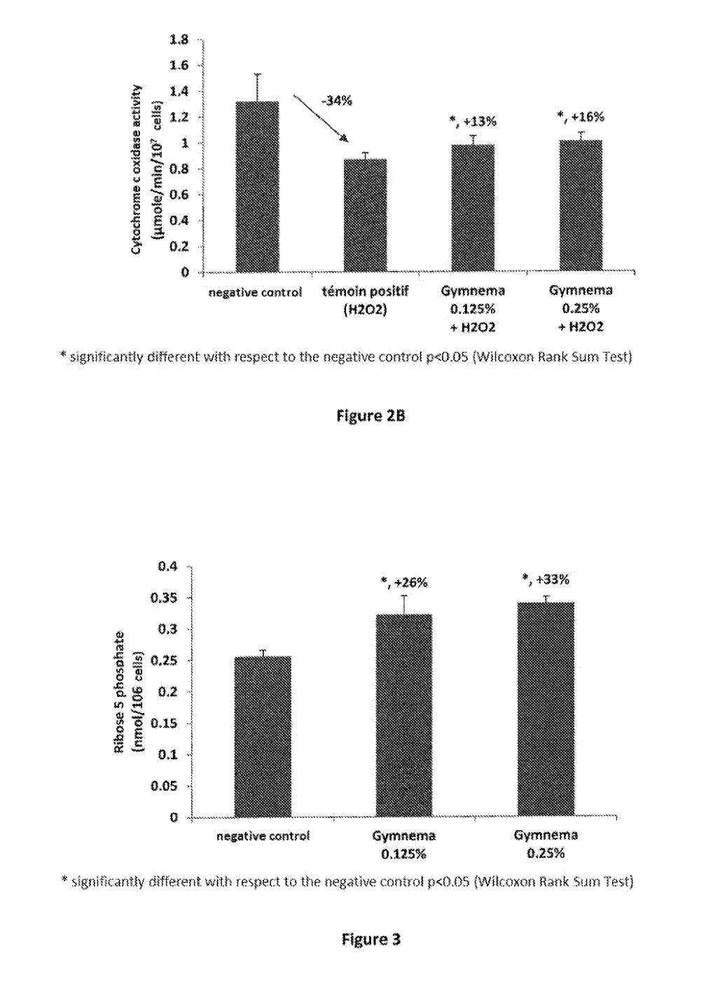

The examples make reference to the following figures wherein the histograms in FIGS. 1, 2A, 2B and 3 represent, respectively, the ATP content, the cytochrome C oxidase activity with or without H₂O₂ and the ribose-5-phosphate content in an untreated control cell culture or treated with 0.125% or 0.25% Gymnema sylvestre leaf extract.

In the set of examples hereinafter, the test product is a Gymnema sylvestre leaf extract obtained using the hydroalcoholic extraction method described above and then stabilized in glycerin (1.5% dry extract in a 70%/30% glycerin/water mixture).

### EXAMPLE 1

### Study of the Effects of an Extract According to the Invention on ATP Syntheses in Human Keratinocytes in Culture

### Materials and Method

### Lot Composition:

Lot 1: untreated control

Lot 2: treated with Gymnema sylvestre extract diluted to 0.125% in culture medium

Lot 3: treated with Gymnema sylvestre extract diluted to 0.25% in culture medium

### Cell Culture:

The keratinocyte cultures were established using skin from human foreskins collected at circumcisions and were amplified in KGM2 medium (Keratinocyte Growth Medium, KGN2, clonetics) supplemented with insulin, EGF and pituitary extract. The tests were conducted on keratinocytes at the 3^{rd} passage. The cells were inoculated in multi-well dishes at a rate of 1×10⁵ cells. After 24 hours of incubation at 37° C. (5% CO₂), the cells in the exponential growth phase were treated with Gymnema sylvestre extract at the concentrations of 0.125% and 0.25%. After 24 hours of treatment, the cells were sampled and the ATP assay was conducted.

### ATP Assay:

The ATP assay was conducted using the PROMOKINE assay kit (Promocell, France). The colorimetric assay was performed using a spectrophotometer at 570 nm.

### Results

TABLE I

ATP
(nmol/10⁶ cells)

| | | standard | |
|---|---|---|---|
| | mean | deviation | % |
| negative control | 2.55 | 0.18 | / |
| Gymnema 0.125% | 3.04 | 0.14 | +19 |
| Gymnema 0.25% | 3.11 | 0.21 | +22 |

The results obtained demonstrate that Gymnema sylvestre extract at the 0.125% and 0.25% concentrations induced a significant increase in ATP synthesis by 19% and 22% respectively.

### EXAMPLE 2

### Study of the Effects of an Extract According to the Invention on the Activity of Cytochrome C Oxidase, Complex IV of the Respiratory Chain, in Human Keratinocytes in Culture Subjected to Oxidative Stress or Not

### Materials and Method

### Lot Composition:

Lot 1: untreated control

Lot 2: treated with Gymnema sylvestre extract diluted to 0.125% in culture medium

Lot 3: treated with Gymnema sylvestre extract diluted to 0.25% in culture medium

Lot 4: treated with H₂O₂

Lot 5: treated with Gymnema sylvestre extract diluted to 0.125% in culture medium +H₂O₂

Lot 6: treated with Gymnema sylvestre extract diluted to 0.25% in culture medium +H₂O₂

### Cell Culture

The keratinocyte cultures were established using skin from human foreskins collected at circumcisions and were amplified in KGM2 medium (Keratinocyte Growth Medium, KGN2, clonetics) supplemented with insulin, EGF and pituitary extract. The tests were conducted on keratinocytes at the 3^{rd} passage. The cells were inoculated in multi-well dishes at a rate of 1×10⁶ cells. After 24 hours of incubation at 37° C. (5% CO₂), the cells in the exponential growth phase were treated with Gymnema sylvestre extract at the concentrations of 0.125% and 0.25% alone or in the presence of H₂O₂.

### Evaluation of Cytochrome C Oxidase Activity

After 24 hours of treatment, the cells were sampled and then washed and resuspended in cold PBS. The evaluation of the cytochrome C oxidase activity was then carried out using the analysis kit (Cytochrome C Oxidase Assay Kit—Sigma) according to the protocol specified.

### Results

TABLE II

ATP
(nmol/10⁷ cells)

| | | standard | |
|---|---|---|---|
| | mean | deviation | % |
| negative control | 1.32 | 0.21 | / |
| Gymnema 0.125% | 1.55 | 0.07 | +17 |
| Gymnema 0.25% | 1.63 | 0.05 | +23 |
| positive control | 0.87 | 0.05 | −34 |
| (H₂O₂) | | | |
| Gymnema 0.125% + | 0.98 | 0.07 | +13 |
| H₂O₂ | | | |
| Gymnema 0.25% + | 1.01 | 0.06 | +16 |
| H₂O₂ | | | |

The results obtained revealed that Gymnema sylvestre extract at the 0.125% and 0.25% concentrations stimulated the cytochrome C oxidase activity of human keratinocytes in culture by 17% and 23%, respectively, compared to the negative control.

The treatment of the cells with hydrogen peroxide alone (H₂O₂) induced a significant decrease in cytochrome C oxidase activity (−34%). In the presence of H₂O₂, Gymnema sylvestre extract at the 0.125% and 0.25% concentrations restored the cytochrome C oxidase activity (+13% and +16%).

In conclusion, Gymnema sylvestre extract has a significant effect on the regulation of metabolic and energy processes in human keratinocytes in culture.

### EXAMPLE 3

### Study of the Effects of an Extract According to the Invention on the Pentose Pathway in Human Keratinocytes In Culture Using the Ribose-5-Phosphate Assay

### Materials and Method

### Lot Composition:

Lot 1: untreated control

Lot 2: treated with Gymnema sylvestre extract diluted to 0.125% in culture medium

Lot 3: treated with Gymnema sylvestre extract diluted to 0.25% in culture medium

### Cell Culture

The method used was that of enzymatic digestion suitable for obtaining primary fibroblast cultures from a human skin biopsy. The cells obtained were amplified in an RPMI 1640 medium supplemented with calf serum, L-glutamine and antibiotics. The tests were conducted on keratinocytes at the 3^{rd} passage. The cells were inoculated in multi-well dishes at a rate of 1×10⁵ cells. After 24 hours of incubation at 37° C. (5% CO₂), the cells in the exponential growth phase were treated with Gymnema sylvestre extract at the concentrations of 0.125% and 0.25%.

### Ribose-5-Phosphate Assay

After 24 hours of treatment, the cells were sampled and the cell sediments were sonicated for 30 s in an ice bath and centrifuged for 5 min at 11,000 g at 10° C. The supernatants were then transferred to new tubes. The residual enzymatic activity was stopped by adding 50 μl of perchloric acid (2.5%) containing 3 μmol/l of IS to the samples which were subsequently stored at −20° C. for at least 30 min for deproteinization. Subsequently, 15 μl of phosphate buffer (pH 11.5; 1 mol/l) was added to neutralize the solution and the samples were centrifuged for 5 min at 21,000 g at 4° C. The supernatants were transferred to glass vials which were stored at −20° until injection. A series of standard solutions was prepared and consists respectively of 0.625; 1.25; 2.5; 3.75 and 6.25 μmol/l of ribose-5-phosphate. The series of standard solutions was treated in the same way as the samples. The ribose-5-phosphate content assay was performed by LC-MS/MS.

### Results

TABLE III

Ribose 5 phosphate
(nmol/10⁶ cells)

| | | standard | |
|---|---|---|---|
| | mean | deviation | % |
| negative control | 0.256 | 0.01 | / |
| Gymnema 0.125% | 0.322 | 0.03 | +26 |
| Gymnema 0.25% | 0.34 | 0.01 | +33 |

The results obtained demonstrate that Gymnema sylvestre extract at the 0.125% and 0.25% concentrations induced a significant increase in ribose-5-phosphate synthesis by 26% and 33% respectively.

In conclusion, Gymnema sylvestre extract has a significant effect on the regulation of metabolic and energy processes in human keratinocytes and fibroblasts in culture.

### EXAMPLE 4

### Compositions

### SERUM %

CARBOMER . . . 0.60
CELLULOSE GUM . . . 0.20
PENTYLENE GLYCOL . . . 2.00
TROMETHAMINE . . . 0.52
GLYCERIN & AQUA & PEG-8 & CAPRYLYL GLYCOL & SODIUM POLYACRYLATE . . . 5.00
AQUA & GLYCERIN & GLYCERYL ACRYLATE/ACRYLIC ACID & COPOLYMER . . . 10.00
GYMNEMA SYLVESTRE . . . 3.00
PRESERVATIVES . . . QS
CHELATING AGENT . . . QS
FRAGRANCE . . . QS
DEIONIZED WATER . . . QS 100

### MOISTURIZING CREAM %

CETEARYL ALCOHOL & SODIUM LAURYL SULFATE & SODIUM CETEARYL SULFATE . . . 1.70
HYDROGENATED POLYISOBUTENE . . . 7.00
ISONONYL ISONONANOATE . . . 5.00
GLYCERYL STEARATE SE . . . 5.60
C12-15 ALKYL BENZOATE . . . 3.50
PEG-20 METHYL GLUCOSE & SESQUISTEARATE . . . 0.50
GLYCERIN . . . 3.00
CARBOMER . . . 0.25
SILICA . . . 0.15
MICA . . . 0.10
TALC . . . 0.50
GLYCERIN & AQUA & PEG-8 & CAPRYLYL GLYCOL & SODIUM POLYACRYLATE . . . 5.00
DIMETHICONE & DIMETHICONOL . . . 0.15
TOCOPHEROL ACETATE . . . 0.20
GYMNEMA SYLVESTRE . . . 1.00
PRESERVATIVES . . . QS
CHELATING AGENT . . . QS
FRAGRANCE . . . QS
DEIONIZED WATER . . . QS 100

### MOISTURIZING GEL %

TAPIOCA STARCH . . . 1.00
AMMONIUM & ACRYLOYLDIMETHYLTAURATE/VP & COPOLYMER . . . 1.00
GLYCOLS . . . 5.00
ETHYLHEXYLGLYCERIN . . . 0.20
AQUA & GLYCERIN & GLYCERYL ACRYLATE/ACRYLIC ACID & COPOLYMER . . . 5.00
CAPRYLIC/CAPRIC TRIGLYCERIDE . . . 1.50
CYCLOMETHICONE . . . 5.00
PHENYL TRIMETHICONE . . . 1.00
CYCLOPENTASILOXANE & PEG/PPG-18/18 DIMETHICONE . . . 1.00
TOCOPHEROL ACETATE . . . 0.20
GYMNEMA SYLVESTRE . . . 1.00
TETRASODIUM EDTA . . . 0.05
PRESERVATIVES . . . QS
FRAGRANCE . . . QS
DEIONIZED WATER . . . QS 100

1. A method of activating energy-producing metabolic pathways of skin cells in a subject, comprising
applying a Gymnema sylvestre extract or a cosmetic composition comprising the Gymnema sylvestre extract to skin cells of the subject.

2. The method according to claim 1, wherein the Gymnema sylvestre extract is a leaf extract.

3. The method according to claim 1, wherein the Gymnema sylvestre extract is a hydroalcoholic extract.

4. The method according to claim 3, wherein the cosmetic composition further comprises glycerin.

5. The method according to claim 1, wherein said cosmetic composition comprises 0.01 to 10% Gymnema sylvestre extract by weight of dry matter with respect to the total weight of the composition.

6. The method according to claim 5, wherein said composition further comprises one or a plurality of formulation agents or additives selected from the group consisting of softeners, colorants, film-forming agents, surfactants, fragrances, preservatives, emulsifiers, oils, glycols, UV filters, and vitamins.

7. The method according to claim 1, wherein the method increases Adenosine triphosphate (ATP) synthesis.

8. The method according to claim 1, wherein the method stimulates cytochrome C oxidase activity.

9. The method according to claim 1, wherein the method induces ribose-5-phosphate synthesis.

10. The method according to claim 1, wherein the method tones the skin cells and/or stimulates metabolism of the skin cells.

11. The method according to claim 1, wherein the method improves skin cell function, and improves homeostasis, renewal and repair of the skin cell function.

12. The method according to claim 1, wherein the method energizes, tones, revitalizes, or detoxifies skin of the subject, enhances color and/or radiance of complexion of the skin, reduces appearance of dull and/or lackluster skin, repairs signs of fatigue on facial skin of the subject, protects the skin from environmental factors.

13. The method according to claim 1, wherein the method tones facial skin of the subject and the contour of the eyes, energizes tired eyes, reduces bags and shadows under the eyes, and restores a relaxed and rested expression on the face.
